(19) United States
(12) Patent Application Publication
(10) Pub. No.: US 2026/0091157 A1
(43) Pub. Date: Apr. 2, 2026

(54) INJECTABLE AND TEMPERATURE-RESPONSIVE ENHANCED COMPOSITE DUAL-NETWORK MAGNETIC PARTICLE GEL, AND PREPARATION METHOD AND USE THEREOF

(71) Applicant: Hefei University of Technology, Hefei (CN)

(72) Inventors: Yang LU, Hefei (CN); Huai YANG, Hefei (CN); Xu YAN, Hefei (CN); Chaowei LU, Hefei (CN); Haojie LU, Hefei (CN); Xi JING, Hefei (CN)

(73) Assignee: Hefei University of Technology, Hefei (CN)

(21) Appl. No.: 19/414,178

(22) Filed: Dec. 9, 2025

(30) Foreign Application Priority Data
Jul. 8, 2025 (CN) 202510932978.X

(51) Int. Cl. A61L 24/10; A61L 24/00; A61L 24/02

(52) U.S. Cl. CPC A61L 24/104; A61L 24/001; A61L 24/0031; A61L 24/02; A61L 2400/12

(57) ABSTRACT

An injectable and temperature-responsive enhanced composite dual-network magnetic particle gel is a hydrogel, including a particle gel network formed by gelatin nanoparticles and Fe₃O₄ nanoparticles via an electrostatic interaction and a poly(N-isopropylacrylamide) (PNIPAM)-based temperature-sensitive polymer gel network. At room temperature, the hydrogel exhibits excellent shear-thinning and self-healing properties, and is easily injectable through needles and microcatheters. When raised to a human body temperature (37° C.), the temperature-sensitive polymer undergoes phase transition cross-linking, enhancing mechanical properties of the gel network. The hydrogel is injectable in vitro and exhibits temperature-responsive strengthening in vivo, meeting the requirements of a vascular interventional embolization material. The hydrogel can also generate magnetothermal heating under an alternating magnetic field, thereby achieving the embolization combined with magnetothermal therapy for liver cancer. The preparation method is simple, with high biocompatibility and a great potential for clinical applications.

### CROSS-REFERENCE TO RELATED APPLICATIONS

This application claims priority to Chinese Patent Application No. 202510932978.X with a filing date of Jul. 8, 2025. The content of the aforementioned application, including any intervening amendments thereto, is incorporated herein by reference.

### TECHNICAL FIELD

The present disclosure relates to the technical field of hydrogels, and in particular to an injectable and temperature-responsive enhanced composite dual-network magnetic particle gel, and a preparation method and use thereof.

### BACKGROUND

Transcatheter arterial embolization (TAE) is an important means for treating unresectable liver cancer. TAE can precisely deliver embolic agents to tumor-feeding arteries through microcatheters. This process cuts off the oxygen and nutrient supply to the lesion, implementing a “starvation therapy” that induces ischemic damage and even necrosis in the tumor. Traditional embolic agents are divided into two categories: solid ones such as polyvinyl alcohol (PVA) microspheres and gelatin sponge particles, and liquid ones such as iodinated oil and n-butyl cyanoacrylate (n-BCA). Each of the two categories has its own advantages and disadvantages. Specifically, PVA microspheres are outstanding in vascular embolization due to their stable embolic effect and durability. However, microspheres with fixed sizes are difficult to fully adapt to target vascular areas with diverse shapes. There is also a risk of microsphere aggregation, which may block the catheter and proximal large vessels, increasing treatment difficulty. In contrast, iodinated oil, as a liquid embolic agent, exhibits excellent fluidity due to its low viscosity. This substance can easily pass through peripheral vascular networks and penetrate deep into tumor vessels, achieving deep embolization. However, the efficacy of iodinated oil is easily affected by blood flow flushing and the body's clearance mechanisms. This may lead to loss of iodinated oil and recanalization of tumor vessels, thereby subsequently inducing tumor recurrence and growth.

Recently, shear-thinning hydrogels have attracted much attention in the field of vascular embolic agents due to their unique properties. During injection, they exhibit fluid-like smoothness in the catheter. Once the stress is removed, they quickly return to the original gel state. Particle gels, as a type of shear-thinning hydrogel, are usually formed by micron-or nano-sized particles dispersed in a liquid. The particle gels form a three-dimensional network gel structure through non-covalent interactions, including van der Waals forces, electrostatic interactions, hydrophobic interactions, and steric hindrance. Thus, they possess excellent shear-thinning and self-healing properties. The physical and chemical characteristics of particle gels can be precisely controlled by finely tuning the composition, size, and concentration of particles, thereby adapting to diverse application scenarios. Currently, particle gels have been widely studied in the biomedical field. For example, particle gels, as drug delivery systems, have significant abilities such as controlling drug release, improving bioavailability, and enhancing therapeutic efficacy. Moreover, due to their shear-thinning characteristics, particle gels can be easily delivered through clinical catheters, showing a great potential as embolic agents. However, a key challenge in the in vivo application of particle gels is stability. This directly affects the long-term effect in embolization therapy and the reduction of ectopic embolization risk. Therefore, it is an important issue that needs to be solved urgently to enhance the in vivo stability of particle gels.

Although covalent cross-linking technology can significantly improve the mechanical strength of particle hydrogels, this enhancement generally sacrifices injectability. It makes the hydrogels difficult to deliver smoothly into blood vessels through microcatheters. Additionally, the added chemical cross-linking agents may reduce the biosafety of the hydrogels. An intravascular in situ gel enhancement strategy involves introducing a second network inside the particle hydrogel in situ. This process can be achieved by using a double syringe or in situ polymerization of monomers in a particle network to realize in situ cross-linking gelation, thereby enhancing the mechanical properties. This strategy not only maintains the injectability of particle hydrogels, but also gives particle hydrogels sufficient mechanical strength to resist blood flow, ensuring the stability and durability of embolization. However, this strategy also faces challenges including uneven mixing that may result from double-syringe operation, and toxicity issues that may be caused by in situ polymerization or chemical cross-linking. Given the urgent need for safer and more efficient gel enhancement technologies, in situ physical cross-linking is receiving widespread attention as a mild strategy. This method induces the formation of a gel network by adjusting external conditions (such as temperature and pH). This method aims to construct a feasible approach for greatly improving the mechanical properties of particle gels while significantly reducing their potential toxicity. In recent years, in situ gelling hydrogels have attracted much attention. Especially temperature-sensitive hydrogels such as poly(N-isopropylacrylamide) (PNIPAM)-based hydrogels show a great potential as embolic agents. These hydrogels can be easily delivered in liquid form, and rapidly transform into solid gels at body temperature, thereby exhibiting excellent application prospects. However, effectively controlling this transformation process still faces challenges. On one hand, an excessively high gelation speed may lead to catheter blockage, increasing the complexity of surgical operations. On the other hand, an insufficient gelation speed may make the gel unable to effectively resist blood flow flushing, affecting the embolization effect. Therefore, it has become the focus of current research to ensure that the gelation is both rapid and controllable, while avoiding the above adverse factors.

Through long-term research and practices of the inventors, the present disclosure is provided to overcome the above defects.

### SUMMARY OF PRESENT INVENTION

An objective of the present disclosure is to address the issue where particle gels, when used as vascular embolic agents, can be delivered to the interior of a tumor via a catheter but are prone to fragmentation under continuous vascular pulsatile pressure and cannot stably embolize blood vessels. An objective of the present disclosure is to provide an injectable and temperature-responsive enhanced composite dual-network magnetic particle gel, and a preparation method and use thereof.

In order to achieve the above objective, the present disclosure provides a preparation method of an injectable and temperature-responsive enhanced composite dual-network magnetic particle gel, including the following steps:
S1, dissolving a temperature-sensitive polymer separately in a solution of gelatin nanoparticles and a solution of Fe₃O₄ nanoparticles in an ice bath, and mixing two obtained solutions to obtain a reaction system;
S2, adding a glucono-δ-lactone (GDL) powder to adjust a pH of the reaction system to less than 7, such that a surface charge of the gelatin nanoparticles changes from negative to positive while a surface charge of the Fe₃O₄ nanoparticles remains negative, and an electrostatic interaction occurs between the two types of nanoparticles to achieve assembly, thereby forming a primary gel network;
S3, heating the reaction system to 37° C., such that the temperature-sensitive polymer dispersed in the primary gel network undergoes hydrophilic-to-hydrophobic phase transition cross-linking to form a secondary gel network, thereby obtaining the injectable and temperature-responsive enhanced composite dual-network magnetic particle gel.

In the present disclosure, a preparation process of the gelatin nanoparticles in the step S1 includes the following steps:
S11, dissolving a gelatin powder in deionized water, adding acetone, allowing an obtained mixture to stand at room temperature for 1 h, discarding an obtained supernatant, redissolving an obtained lower gelatin precipitate in the deionized water, and conducting freeze-drying to obtain purified gelatin, where the gelatin powder is added at a mass of 1 g to 50 g, and the deionized water and the acetone each are added at a volume of 50 mL to 500 mL;
S12, dissolving the purified gelatin obtained in the step S11 in the deionized water, adding dilute hydrochloric acid to adjust a pH of an obtained solution to 2.3, dropwise adding the acetone under vigorous stirring, adding glutaraldehyde, stirring overnight, adding an aqueous solution of glycine to an obtained gelatin dispersion, stirring for 1 h, and conducting centrifugation and washing to obtain the gelatin nanoparticles, where the purified gelatin and the glycine are added at masses of 1 g to 10 g and 1 g to 5 g, respectively, and the deionized water, the acetone, and the glutaraldehyde are added at volumes of 50 mL to 100 mL, 200 mL to 400 mL, and 0.1 mL to 2 mL, respectively.

In the present disclosure, a preparation process of the Fe₃O₄ nanoparticles in the step S1 includes the following steps: dissolving FeCl₃·6H₂O in a mixed solvent of ethylene glycol (EG) and diethylene glycol (DEG), adding polyacrylic acid (PAA) and stirring to allow a reaction for 1 h, adding sodium acetate (NaAc) and stirring to allow a reaction for 1 h, thereby obtaining a viscous precursor solution; and placing the viscous precursor solution in a 50 mL hydrothermal autoclave, conducting a hydrothermal reaction at 200° C. for 12 h, cooling, subjecting an obtained reaction product to centrifugation and washing with the deionized water 3 times, followed by freeze-drying to obtain the Fe₃O₄ nanoparticles.

In the present disclosure, the FeCl₃·6H₂O, the PAA, and the NaAc are added at masses of 1 g to 10 g, 0.1 g to 1 g, and 1 g to 10 g, respectively, and the EG and the DEG are added at volumes of 1 mL to 20 mL and 1 mL to 30 mL, respectively.

In the present disclosure, a preparation process of the temperature-sensitive polymer in the step S1 includes the following steps: dissolving polyethylene glycol (PEG) and triethylamine (TEA) in dichloromethane (DCM), introducing argon for protection, dropwise adding 2-bromoisobutyryl bromide at 0° C., stirring to allow a reaction at room temperature for 24 h, and subjecting an obtained reaction product to dialysis and freeze-drying in sequence to obtain an atom transfer radical polymerization initiator; and dissolving the atom transfer radical polymerization initiator in an isopropanol (IPA)/water mixed solvent, adding N-isopropylacrylamide (NIPAM), n-butyl acrylate (nBA), and cuprous chloride (CuCl) to allow full dissolution, introducing the argon for protection, conducting three freeze-thaw cycles under argon protection to allow oxygen removal, injecting tris[2-(dimethylamino)ethyl]amine via a microsyringe to allow polymerization at room temperature for 72 h, subjecting an obtained polymerized product to separation with a neutral alumina column, and subjecting an obtained separated product to dialysis and freeze-drying in sequence to obtain the temperature-sensitive polymer.

In the present disclosure, PEG, TEA, and 2-bromoisobutyryl bromide are added at masses of 10 g to 30 g, 1 g to 10 g, and 2 g to 8 g, respectively.

In the present disclosure, the atom transfer radical polymerization initiator, the NIPAM, the nBA, and the CuCl are added at masses of 0.1 g to 0.5 g, 1 g to 10 g, 0.1 g to 0.5 g, and 10 mg to 50 mg, respectively, and tris[2-(dimethylamino)ethyl]amine is added at a volume of 50μL to 200μL.

In the present disclosure, the gelatin nanoparticles, the Fe₃O₄ nanoparticles, and the temperature-sensitive polymer have solid contents of 4% to 8% weight by volume (w/v), 2% to 6% w/v, and 5% to 15% w/v, respectively, and the gelatin nanoparticles and the Fe₃O₄ nanoparticles each have a particle size of 100 nm to 300 nm in the injectable and temperature-responsive enhanced composite dual-network magnetic particle gel obtained in the step S3.

The present disclosure further provides an injectable and temperature-responsive enhanced composite dual-network magnetic particle gel prepared by the preparation method and use of the injectable and temperature-responsive enhanced composite dual-network magnetic particle gel in preparation of a drug for embolization combined with magnetothermal therapy for liver cancer.

Compared with the prior art, the present disclosure has the following beneficial effects:
1. In the present disclosure, the injectable and temperature-responsive enhanced composite dual-network magnetic particle gel forms a primary gel network through electrostatic interactions between the two types of nanoparticles at room temperature. The primary gel network exhibits excellent injectability and self-healing properties. When the hydrogel enters the human body and responds to body temperature (approximately 37° C.), the temperature-sensitive polymer between the two types of nanoparticles undergoes hydrophilic-to-hydrophobic phase transition cross-linking, forming a secondary gel network. The secondary gel network significantly enhances the stability and strength of the entire gel network system, meeting the performance requirements for clinical vascular embolic agents.
2. In the present disclosure, the injectable and temperature-responsive enhanced composite dual-network magnetic particle gel possesses stable magnetothermal properties, thereby enabling effective thermal killing of tumor cells.
3. In the present disclosure, the injectable and temperature-responsive enhanced composite dual-network magnetic particle gel can be accurately delivered to the deep blood vessels of liver cancer through a catheter. This hydrogel embolizes the tumor's feeding arteries and cuts off the supply of oxygen and nutrients. Simultaneously, when combined with magnetothermal effects, the hydrogel effectively inhibits tumor progression. This process achieves a synergistic therapeutic effect of embolization combined with magnetothermal therapy.
4. In the present disclosure, the injectable and temperature-responsive enhanced composite dual-network magnetic particle gel offers the advantages of no pollution, desirable biocompatibility, and low cytotoxicity. As a result, the hydrogel is a highly safe material with stable structure and strong practicality. The hydrogel can hold a significant potential for clinical and market application.

### BRIEF DESCRIPTION OF THE DRAWINGS

FIG. 1 shows a schematic diagram of the preparation principle of the injectable and temperature-responsive enhanced composite dual-network magnetic particle gel of the present disclosure;

FIG. 2 shows a scanning electron microscope (SEM) image of the gelatin nanoparticles;

FIG. 3 shows a SEM image of the Fe₃O₄ nanoparticles;

FIG. 4 shows a SEM image of a hydrogel formed by the gelatin nanoparticles and the Fe₃O₄ nanoparticles through electrostatic interactions;

FIG. 5 shows a SEM image of the injectable and temperature-responsive enhanced composite dual-network magnetic particle gel;

FIG. 6 shows an elemental distribution image of the injectable and temperature-responsive enhanced composite dual-network magnetic particle gel prepared in Example 1 of the present disclosure;

FIG. 7 shows an X-ray Diffraction (XRD) pattern of the injectable and temperature-responsive enhanced composite dual-network magnetic particle gel prepared in Example 1 of the present disclosure;

FIG. 8 shows a bar chart of the Zeta potential of the constituent units of the injectable and temperature-responsive enhanced composite dual-network magnetic particle gel prepared in Example 1 of the present disclosure at different pH values;

FIG. 9 shows a compression test and corresponding infrared thermal imaging of the injectable and temperature-responsive enhanced composite dual-network magnetic particle gel prepared in Example 1 of the present disclosure, where (I) shows the compression test at 25° C., and (II) shows the compression test at 37° C.;

FIG. 10 shows a modulus-temperature curve of Hydrogel 1 to Hydrogel 6 of the present disclosure;

FIG. 11 shows a viscosity-shear rate curve of Hydrogel 1 to Hydrogel 4 of the present disclosure;

FIG. 12 shows a bar chart of the injection force of Hydrogel 3 of the present disclosure through a 26 Gauge (26G) needle at ambient temperatures of 25° C. and 37° C., respectively;

FIG. 13 shows a modulus-frequency curve of Hydrogel 3 of the present disclosure;

FIG. 14 shows a thixotropy test of Hydrogel 3 of the present disclosure under repeated oscillation cycles at low strain (0.01%) and high strain (500%);

FIG. 15 shows a bar chart of the injection force of Hydrogel 3 of the present disclosure through different types of catheters at different injection rates;

FIG. 16 shows a modulus-temperature curve of the temperature-sensitive hydrogel prepared in the Comparative Example of the present disclosure;

FIG. 17 shows a viscosity bar chart of the temperature-sensitive hydrogel prepared in the Comparative Example of the present disclosure;

FIG. 18 shows a gel time bar chart of the temperature-sensitive hydrogel prepared in the Comparative Example of the present disclosure;

FIG. 19 shows an injection force curve of the temperature-sensitive hydrogel prepared in the Comparative Example of the present disclosure;

FIG. 20 shows a schematic diagram of the hydrogel embolization pressure test;

FIG. 21 shows embolization pressure curves of the hydrogels prepared in Example 1 and the Comparative Example of the present disclosure;

FIG. 22 shows corresponding embolization pressure bar charts of the hydrogels prepared in Example 1 and the Comparative Example of the present disclosure;

FIG. 23 shows an intravascular simulator;

FIG. 24 shows images of the embolization simulation process of Hydrogel 1 prepared in Comparative Example 1 of the present disclosure in an intravascular simulator at 37° C., where the black substance in the simulated tumor tissue represents the hydrogel;

FIG. 25 shows images of the embolization simulation process of the injectable and temperature-responsive enhanced composite dual-network magnetic particle gel prepared in Example 1 of the present disclosure in an intravascular simulator, where the black substance in the simulated tumor tissue represents the hydrogel;

FIG. 26 shows digital subtraction angiography (DSA) images of the rabbit renal artery and branches pre-and post-kidney embolization with the injectable and temperature-responsive enhanced composite dual-network magnetic particle gel prepared in Example 1 of the present disclosure, where the black dashed box represents the embolized right kidney, and the arrow indicates the 4 French (4F) catheter;

FIG. 27 shows an X-ray fluoroscopy image of the injectable and temperature-responsive enhanced composite dual-network magnetic particle gel prepared in Example 1 of the present disclosure in the embolized kidney, where the dashed box represents the embolized right kidney, the right renal vessels are filled with hydrogel, and the arrow indicates the 2.6F microcatheter;

FIG. 28 shows cross-sectional computed tomography (CT) images of the rabbit kidney on day 7 and day 28 post-kidney embolization with the injectable and temperature-responsive enhanced composite dual-network magnetic particle gel prepared in Example 1 of the present disclosure, where the positive control group is not embolized, and the dashed circle indicates the location of the kidney;

FIG. 29 shows physical images of the rabbit kidney pre-and post-embolization with the injectable and temperature-responsive enhanced composite dual-network magnetic particle gel prepared in Example 1 of the present disclosure, where the dashed box represents the embolized right kidney;

FIG. 30 shows H&E staining of tissue sections of the rabbit kidney embolized with the injectable and temperature-responsive enhanced composite dual-network magnetic particle gel prepared in Example 1 of the present disclosure, where the dashed circle represents the glomerulus;

FIG. 31 shows Prussian blue staining of tissue sections of the rabbit kidney embolized with the injectable and temperature-responsive enhanced composite dual-network magnetic particle gel prepared in Example 1 of the present disclosure, where the arrow indicates the hydrogel;

FIG. 32 shows infrared thermal images over time of a spherical agarose simulated tissue loaded with the injectable and temperature-responsive enhanced composite dual-network magnetic particle gel prepared in Example 1 of the present disclosure under different magnetic field strengths;

FIG. 33 shows curves of the surface temperature versus time of a spherical agarose simulated tissue loaded with the injectable and temperature-responsive enhanced composite dual-network magnetic particle gel prepared in Example 1 of the present disclosure under different magnetic field strengths;

FIG. 34 shows curves of the internal temperature versus time of a spherical agarose simulated tissue loaded with the injectable and temperature-responsive enhanced composite dual-network magnetic particle gel prepared in Example 1 of the present disclosure under different magnetic field strengths, where the inset shows a picture of the internal temperature of the sphere tested by an optical fiber temperature sensor;

FIG. 35 shows DSA images of a rabbit liver cancer pre-and post-embolization with the injectable and temperature-responsive enhanced composite dual-network magnetic particle gel prepared in Example 1 of the present disclosure;

FIG. 36 shows physical images of the liver 14 days post-embolization combined with magnetothermal therapy for rabbit liver cancer using the injectable and temperature-responsive enhanced composite dual-network magnetic particle gel prepared in Example 1 of the present disclosure, where the control group receives no treatment;

FIG. 37 shows H&E staining of tumor tissues 14 days post-embolization combined with magnetothermal therapy for rabbit liver cancer using the injectable and temperature-responsive enhanced composite dual-network magnetic particle gel prepared in Example 1 of the present disclosure;

FIG. 38 shows Prussian blue staining of tumor tissues 14 days post-embolization combined with magnetothermal therapy for rabbit liver cancer using the injectable and temperature-responsive enhanced composite dual-network magnetic particle gel prepared in Example 1 of the present disclosure, where the arrow indicates the hydrogel.

### DETAILED DESCRIPTION OF THE EMBODIMENTS

The above and other technical features and advantages of the present disclosure will be described below in more details in connection with the accompanying drawings.

### Example 1

In this example, the injectable and temperature-responsive enhanced composite dual-network magnetic particle gel is prepared according to the following steps:

### 1. Preparation of Gelatin Nanoparticles

In step (1), 25 g of gelatin powder is dissolved in 1 L of deionized water (50° C.). Then, 500 mL of acetone is added, and the mixture is left to stand at room temperature for 1 h. The supernatant is discarded, and the lower gelatin precipitate is redissolved in deionized water. Finally, the purified gelatin is obtained by freeze-drying.

In step (2), 3.75 g of the purified gelatin obtained in step (1) is dissolved in 30 mL of deionized water (50° C.). Dilute hydrochloric acid is added to adjust the pH to 2.3. Then, 225 mL of acetone is added dropwise under vigorous stirring. Subsequently, 580 mL of glutaraldehyde (25 wt%) is added, and a resulting mixture is stirred overnight. Finally, 100 mL of a glycine aqueous solution (3%, w/v) is added to a resulting gelatin dispersion, followed by stirring for 1 h. The gelatin nanoparticles are obtained after centrifugation and washing.

### 2. Preparation of Fe₃O₄ Nanoparticles

1.08 g of FeCl₃·6H₂O is dissolved in a mixed solvent of 10 mL of EG and 20 mL of DEG. Then, 0.1 g of PAA and 6 g of NaAc are added sequentially, with each addition followed by stirring and reaction for 1 h. A resulting viscous precursor solution is placed in a 50 mL hydrothermal autoclave and subjected to hydrothermal reaction at 200° C. for 12 h. After cooling, a resulting product is washed 3 times by centrifugation with deionized water (8,000 revolutions per minute (rpm), 5 min) and then freeze-dried to obtain the Fe₃O₄ nanoparticles.

### 3. Preparation of Temperature-Sensitive Polymer

In step (1), 15 g of PEG and 1.8 g of TEA are dissolved in 150 mL of DCM. Argon is introduced for protection. Then, 3.4 g of 2-bromoisobutyryl bromide is added dropwise at 0° C. A resulting mixture is stirred at room temperature to allow reaction for 24 h, then dialyzed and freeze-dried to obtain an atom transfer radical polymerization initiator.

In step (2), 0.2 g of the atom transfer radical polymerization initiator obtained in step (1) is dissolved in 6 mL of an IPA/water (95/5, V/V) mixed solvent. Then, 2.5 g of NIPAM, 0.16 g of nBA, and 28.6 mg of CuCl are added and fully dissolved. Argon is introduced for protection. After three freeze-thaw cycles under argon protection to allow oxygen removal, 94 μL of tris[2-(dimethylamino)ethyl]amine is injected via a microsyringe. A resulting mixture is subjected to polymerization at room temperature for 72 h. A resulting product is separated using a neutral alumina column, dialyzed, and freeze-dried to obtain the temperature-sensitive polymer.

### 4. Preparation of the Injectable and Temperature-Responsive Enhanced Composite Dual-Network Magnetic Particle Gel

0.2 g of the temperature-sensitive polymer is separately dissolved in 2 mL of a solution of the gelatin nanoparticles (8%, w/v, pH=10) and 2 mL of a solution of the Fe₃O₄ nanoparticles (2%, w/v, pH=10) in an ice bath. Two resulting solutions are then mixed uniformly. 80 mM of GDL powder is added to adjust a pH of a resulting reaction system to an acidic value (pH<7). Finally, the reaction system is heated to 37° C. to obtain the injectable and temperature-responsive enhanced composite dual-network magnetic particle gel, designated as Hydrogel 3.

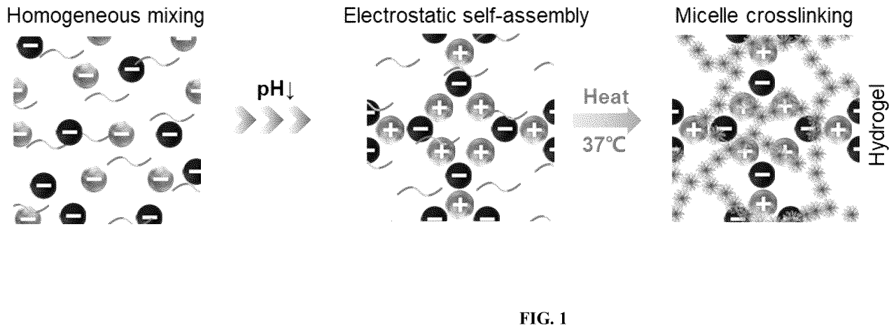

As shown in FIG. 1, the hydrogel undergoes a two-step gelation process. First, at room temperature, after acidification treatment, the surface charge of the gelatin nanoparticles changes from negative to positive. Subsequently, a primary particle gel network is formed with the gelatin nanoparticles and the negatively charged Fe₃O₄ nanoparticles via electrostatic interactions. At this stage, the hydrogel exhibits excellent shear-thinning and self-healing properties, allowing easy delivery through microcatheters. Subsequently, when the hydrogel enters the human body and responds to body temperature, the temperature-sensitive polymer dispersed between the two types of nanoparticles undergoes hydrophilic-to-hydrophobic phase transition cross-linking, forming a secondary polymer gel network. Finally, an injectable and temperature-responsive enhanced composite dual-network magnetic particle gel is obtained.

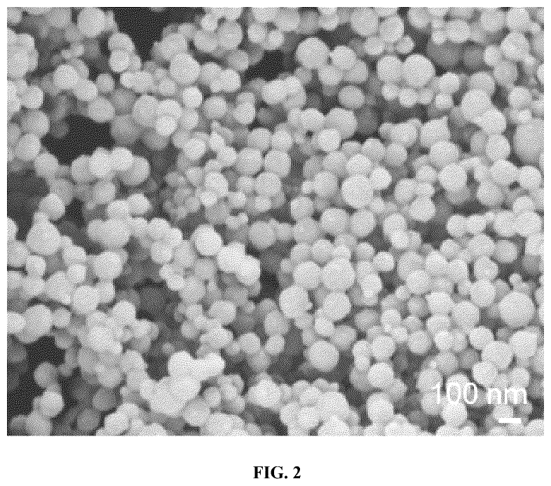

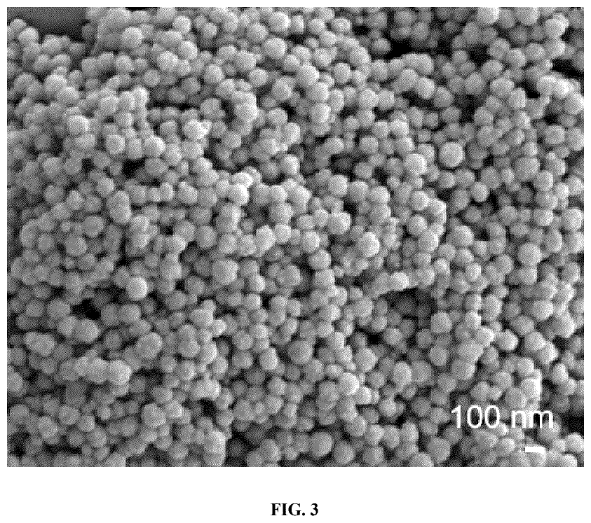

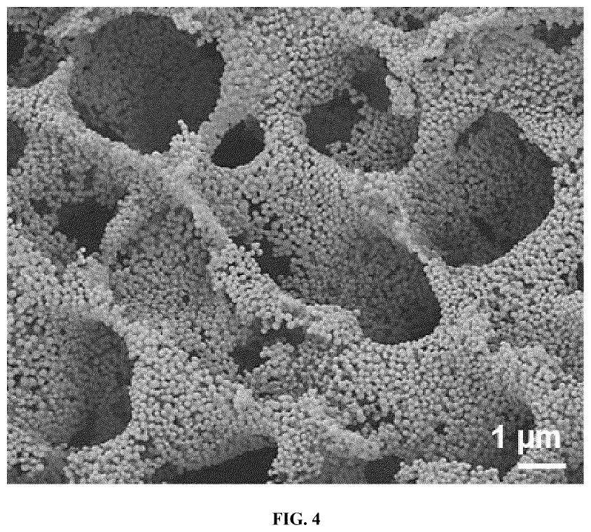

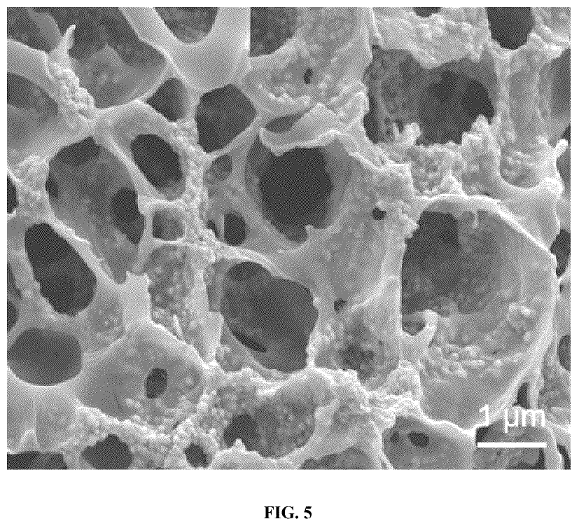

FIG. 2 to FIG. 5 are SEM images (Zeiss Supra 40, Germany) of the injectable and temperature-responsive enhanced composite dual-network magnetic particle gel from Example 1 and its constituent units. FIG. 2 and FIG. 3 show that both the gelatin nanoparticles and Fe₃O₄ nanoparticles exhibit uniform spherical morphology (diameter approximately 100 nm). As shown in FIG. 4, the particle hydrogel formed by the two types of nanoparticles presents a relatively uniform particle network. As shown in FIG. 5, when the temperature-sensitive polymer is incorporated into the particle gel system and the temperature reaches 37° C., the temperature-sensitive polymer infiltrating the interstitial spaces of the particle network forms a secondary gel network, tightly encapsulating the nanoparticles and significantly enhancing the stability of the entire gel network.

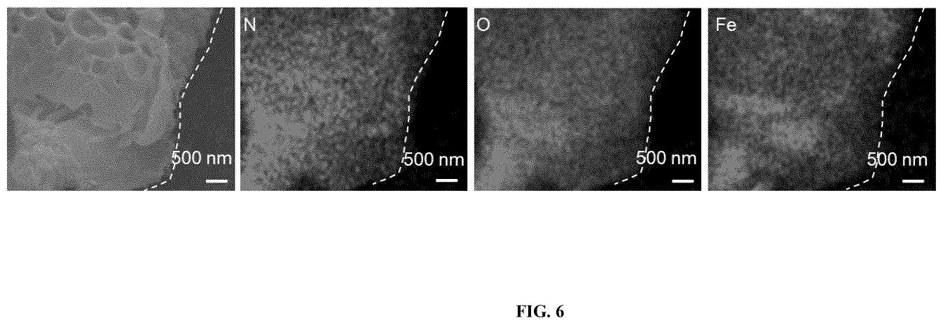

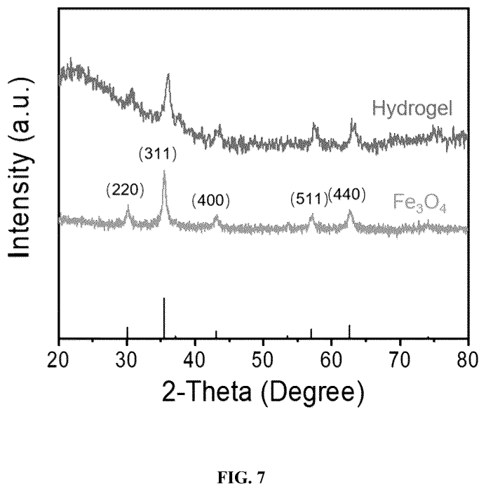

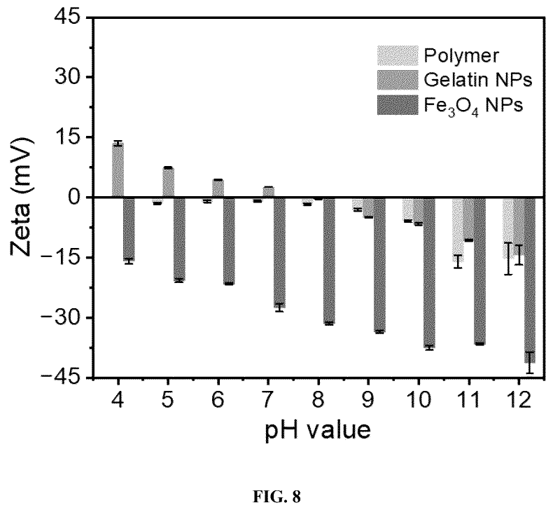

FIG. 6 to FIG. 8 characterize the elemental composition or potential of the injectable and temperature-responsive enhanced composite dual-network magnetic particle gel from Example 1. FIG. 6 shows a uniform distribution of elements such as Fe, N, and O, further confirming the homogeneous mixing of the hydrogel building blocks. The XRD pattern in FIG. 7 shows that the peaks match well with magnetite, indicating the presence of ferric oxide crystals in the composite hydrogel. From FIG. 8, it can be seen that as the pH of reaction system decreases, the surface potential of the gelatin nanoparticles changes from negative to positive, enabling electrostatic assembly with the negatively charged Fe₃O₄ nanoparticles, and this process is almost unaffected by the temperature-sensitive polymer.

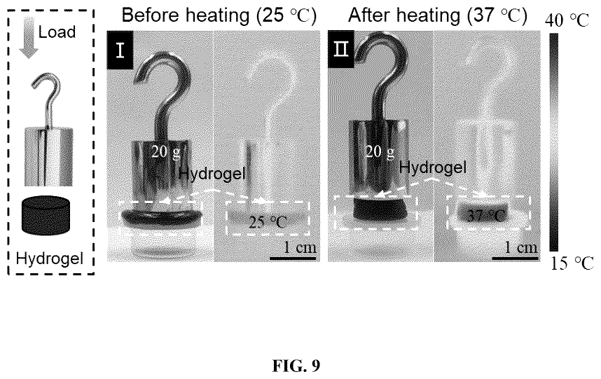

FIG. 9 shows the compression tests and corresponding infrared thermal imaging of the injectable and temperature-responsive enhanced composite dual-network magnetic particle gel prepared in Example 1 at 25° C. and 37° C. The compression experiments compare the anti-deformation performance of the hydrogel before and after heat treatment under the pressure of a 20 g counterweight. At 25° C., the hydrogel exhibits significant compression deformation, reflecting its weaker mechanical strength. When the temperature is raised to 37° C., the degree of compression deformation of the hydrogel is significantly reduced, demonstrating stronger mechanical properties.

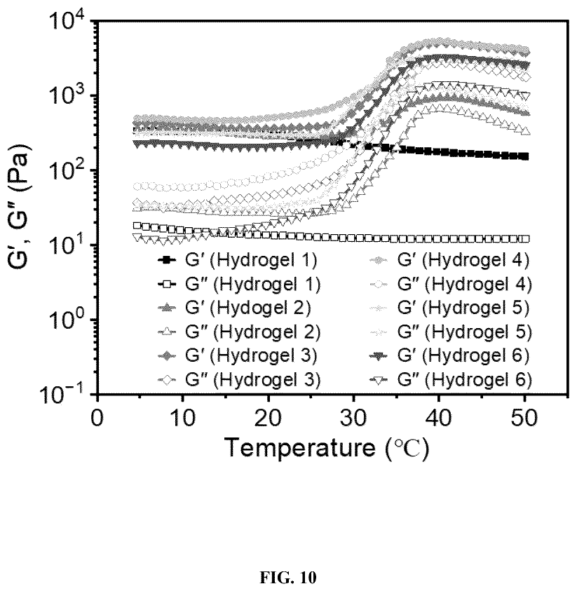

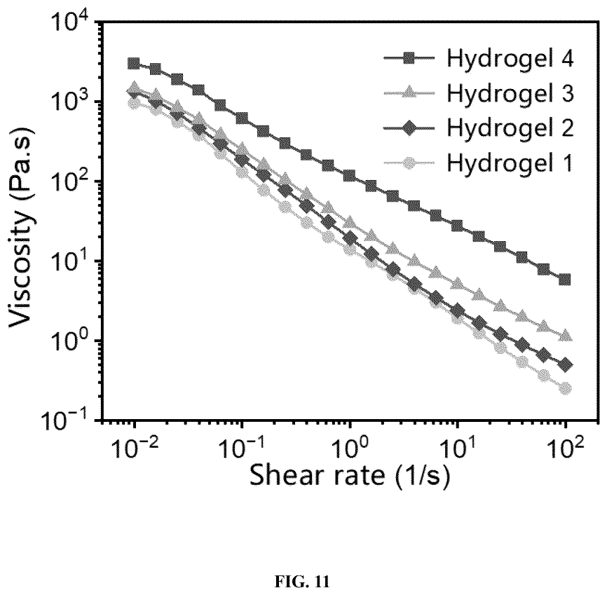

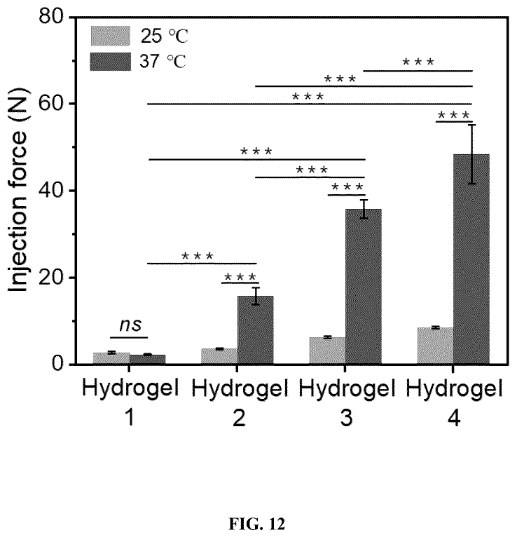

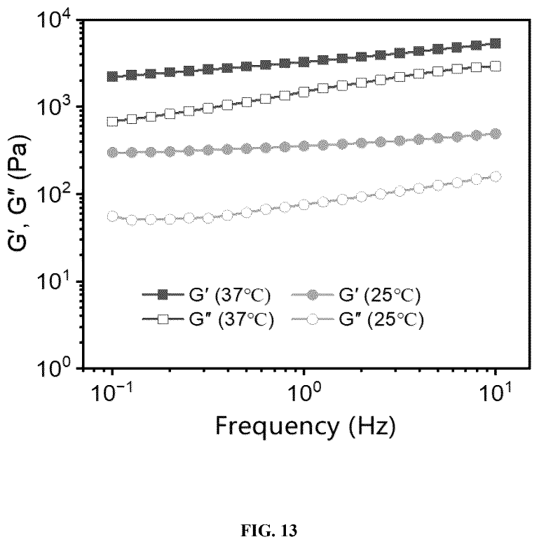

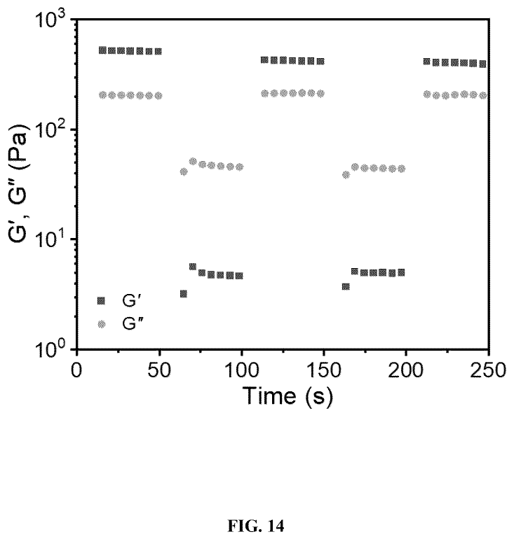

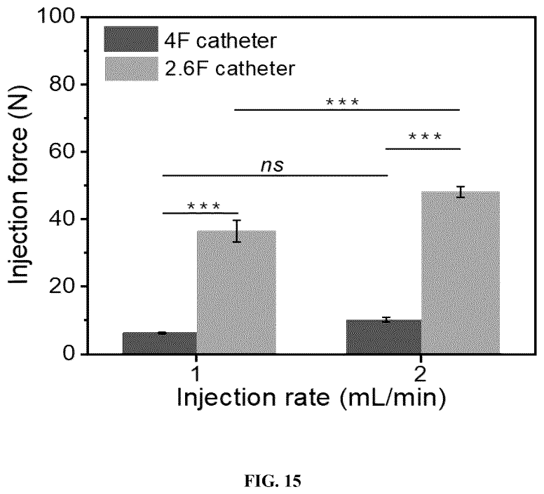

FIG. 10 to FIG. 15 present the rheological and injection performance tests of the injectable and temperature-responsive enhanced composite dual-network magnetic particle gel. From FIG. 10, it can be seen that as the temperature increases, the storage modulus and loss modulus of Hydrogel 3 begin to increase starting at 30° C., with the maximum storage modulus and maximum loss modulus reaching 4,964.90 Pa and 2,721.69 Pa, respectively. This indicates the temperature-sensitive strengthening characteristic of the hydrogel. From FIG. 11, it can be seen that the viscosity of Hydrogel 3 decreases with increasing shear rate, exhibiting shear-thinning behavior, which proves the injectability of the hydrogel. From FIG. 12, it can be seen that when Hydrogel 3 passes through a 26G needle at 25° C. and 37° C., respectively, the injection force difference is significant (increasing from 6.25 N±0.28 N to 35.77 N±2.17 N), further proving the enhanced mechanical properties of the hydrogel after temperature increase. From FIG. 13, it can be seen that Hydrogel 3 exhibits stable solid-like behavior in the frequency range of 0.1-10 Hz. From FIG. 14, it can be seen that Hydrogel 3 at 25° C. shows recoverable modulus over cycles, indicating excellent self-healing properties of the hydrogel. From FIG. 15, it can be seen that the injection force required for the hydrogel to pass through a 4F catheter (inner diameter =0.89 mm, length=100 cm) and a 2.6F catheter (inner diameter=0.53 mm, length=125 cm) at an injection rate of 1 mL/min is within the range suitable for manual operation (6.34 N±0.17 N and 36.50 N±3.33 N, respectively). The above results indicate that the hydrogel meets the performance requirements for vascular embolic agents.

### Example 2

This example differs from Example 1 in that the solid content of the temperature-sensitive polymer is 5% w/v, while all other preparation conditions remain the same. Specifically, 0.1 g of the temperature-sensitive polymer is separately dissolved in 2 mL of a solution of the gelatin nanoparticles (8%, w/v, pH=10) and 2 mL of a solution of the Fe₃O₄ nanoparticles (2%, w/v, pH=10) in an ice bath. Two resulting solutions are then mixed uniformly. 80 mM of GDL powder is added to adjust a pH of a resulting reaction system to an acidic value (pH<7). Finally, the reaction system is heated to 37° C., causing the temperature-sensitive polymer in the particle network to undergo hydrophilic-to-hydrophobic phase transition cross-linking, forming a dual-layer gel network. Therefore, the injectable and temperature-responsive enhanced composite dual-network magnetic particle gel is obtained, designated as Hydrogel 2.

From FIG. 10, it can be seen that as the temperature increases, the storage modulus and loss modulus of Hydrogel 2 begin to increase starting at 30° C., with the maximum storage modulus and maximum loss modulus reaching 925.7 Pa and 657.7 Pa, respectively. This indicates the temperature-sensitive strengthening characteristic of the hydrogel. From FIG. 11, it can be seen that the viscosity of Hydrogel 2 decreases with increasing shear rate, exhibiting shear-thinning behavior, which proves the injectability of the hydrogel. From FIG. 12, it can be seen that when Hydrogel 2 passes through a 26G needle at 25° C. and 37° C., respectively, the injection force difference is significant (increasing from 3.60 N±0.20 N to 15.69 N±1.99 N), further proving the enhanced mechanical properties of the hydrogel after temperature increase. Therefore, the injectable and temperature-responsive enhanced composite dual-network magnetic particle gel prepared in this example exhibits excellent temperature responsiveness, shear-thinning, and injectability. The above results indicate that the hydrogel meets the performance requirements for vascular embolic agents.

### Example 3

This example differs from Example 1 in that the solid content of the temperature-sensitive polymer is 15% w/v, while all other preparation conditions remain the same. Specifically, 0.3 g of the temperature-sensitive polymer is separately dissolved in 2 mL of a solution of the gelatin nanoparticles (8%, w/v, pH=10) and 2 mL of a solution of the Fe₃O₄ nanoparticles (2%, w/v, pH=10) in an ice bath. Two resulting solutions are then mixed uniformly. 80 mM of GDL powder is added to adjust a pH of a resulting reaction system to an acidic value (pH<7). Finally, the reaction system is heated to 37° C., causing the temperature-sensitive polymer in the particle network to undergo hydrophilic-to-hydrophobic phase transition cross-linking, forming a dual-layer gel network. Therefore, the injectable and temperature-responsive enhanced composite dual-network magnetic particle gel is obtained, designated as Hydrogel 4.

From FIG. 10, it can be seen that as the temperature increases, the storage modulus and loss modulus of Hydrogel 4 begin to increase starting at 30° C., with the maximum storage modulus and maximum loss modulus reaching 5,343.37 Pa and 3,157.92 Pa, respectively. This indicates the temperature-sensitive strengthening characteristic of the hydrogel. From FIG. 11, it can be seen that the viscosity of Hydrogel 4 decreases with increasing shear rate, exhibiting shear-thinning behavior, which proves the injectability of the hydrogel. From FIG. 12, it can be seen that when Hydrogel 4 passes through a 26G needle at 25° C. and 37° C., respectively, the injection force difference is significant (increasing from 8.50 N±0.25 N to 48.39 N±6.76 N), further proving the enhanced mechanical properties of the hydrogel after temperature increase. Therefore, the injectable and temperature-responsive enhanced composite dual-network magnetic particle gel prepared in this example exhibits excellent temperature responsiveness, shear-thinning, and injectability. The above results indicate that the hydrogel meets the performance requirements for vascular embolic agents.

### Example 4

This example differs from Example 1 in that the solid contents of the gelatin nanoparticles and Fe₃O₄ nanoparticles are 6% w/v and 4% w/v, respectively, while all other preparation conditions remain the same. Specifically, 0.2 g of the temperature-sensitive polymer is separately dissolved in 2 mL of a solution of the gelatin nanoparticles (6%, w/v, pH=10) and 2 mL of a solution of the Fe₃O₄ nanoparticles (4%, w/v, pH=10) in an ice bath. Two resulting solutions are then mixed uniformly. 80 mM of GDL powder is added to adjust a pH of a resulting reaction system to an acidic value (pH<7). Finally, the reaction system is heated to 37° C., causing the temperature-sensitive polymer in the particle network to undergo hydrophilic-to-hydrophobic phase transition cross-linking, forming a dual-layer gel network. Therefore, the injectable and temperature-responsive enhanced composite dual-network magnetic particle gel is obtained, designated as Hydrogel 5.

The properties of the injectable and temperature-responsive enhanced composite dual-network magnetic particle gel prepared in this example are similar to those in Example 1. From FIG. 10, it can be seen that as the temperature increases, the storage modulus and loss modulus of Hydrogel 5 begin to increase starting at 30° C., with the maximum storage modulus and maximum loss modulus reaching 3,221.49 Pa and 1,292.78 Pa, respectively. This indicates the temperature-sensitive strengthening characteristic of the hydrogel and meets the performance requirements for vascular embolic agents.

### Example 5

This example differs from Example 1 in that the solid contents of the gelatin nanoparticles and Fe₃O₄ nanoparticles are 4% w/v and 6% w/v, respectively, while all other preparation conditions remain the same. Specifically, 0.2 g of the temperature-sensitive polymer is separately dissolved in 2 mL of a solution of the gelatin nanoparticles (4%, w/v, pH=10) and 2 mL of a solution of the Fe₃O₄ nanoparticles (6%, w/v, pH=10) in an ice bath. Two resulting solutions are then mixed uniformly. 80 mM of GDL powder is added to adjust a pH of a resulting reaction system to an acidic value (pH<7). Finally, the reaction system is heated to 37° C., causing the temperature-sensitive polymer in the particle network to undergo hydrophilic-to-hydrophobic phase transition cross-linking, forming a dual-layer gel network. Therefore, the injectable and temperature-responsive enhanced composite dual-network magnetic particle gel is obtained, designated as Hydrogel 6.

The properties of the injectable and temperature-responsive enhanced composite dual-network magnetic particle gel prepared in this example are similar to those in Example 1. From FIG. 10, it can be seen that as the temperature increases, the storage modulus and loss modulus of Hydrogel 6 begin to increase starting at 30° C., with the maximum storage modulus and maximum loss modulus reaching 3,191.31 Pa and 1,381.99 Pa, respectively. This indicates that the hydrogel exhibits the temperature-sensitive strengthening characteristic and meets the performance requirements for vascular embolic agents.

### Comparative Example 1

This comparative example differs from Example 1 in that the solid content of the temperature-sensitive polymer is 0% w/v, while all other preparation conditions remain the same. Specifically, 2 mL of a solution of the gelatin nanoparticles (8%, w/v, pH=10) and 2 mL of a solution of the Fe₃O₄ nanoparticles (2%, w/v, pH=10) are mixed uniformly. 80 mM of GDL powder is added to adjust a pH of a resulting reaction system to an acidic value (pH<7), thereby obtaining an injectable magnetic particle gel (Hydrogel 1).

From FIG. 4, it can be seen that the particle hydrogel formed by the two types of nanoparticles presents a relatively uniform particle network. From FIG. 10, it can be seen that when the temperature increases from 25° C. to 37° C., the storage modulus and loss modulus of Hydrogel 1 slightly decrease from 254.23 Pa and 12.75 Pa to 179.91 Pa and 12.04 Pa, respectively. From FIG. 11, it can be seen that the viscosity of Hydrogel 1 decreases with increasing shear rate, exhibiting shear-thinning behavior, which proves the injectability of the hydrogel. From FIG. 12, it can be seen that the injection force of Hydrogel 1 through a 26G needle does not change significantly at 25° C. and 37° C. The above results indicate that although the hydrogel is injectable through catheters, it has weak mechanical properties and cannot achieve stable intravascular embolization, thereby failing to meet the performance requirements for vascular embolic agents.

### Comparative Example 2

This comparative example adopts the same synthesis method for the temperature-sensitive polymer as that in Example 1. 0.05 g of the temperature-sensitive polymer is dissolved in 1 mL of an aqueous sodium hydroxide solution (pH=10) in an ice bath. Finally, a resulting reaction system is heated to 37° C. to obtain a temperature-sensitive polymer hydrogel, designated as Hydrogel 7.

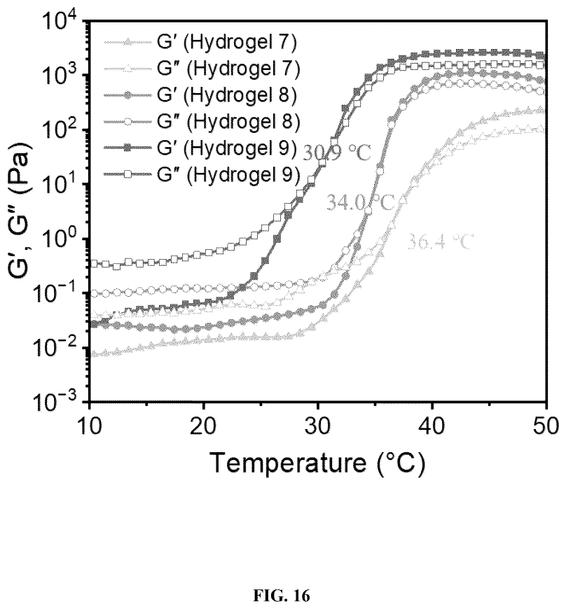

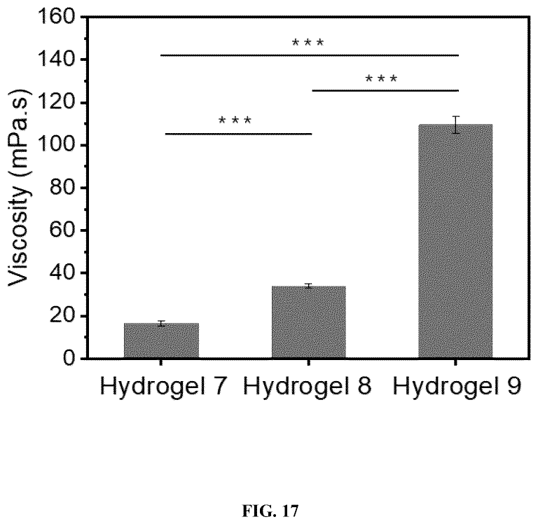

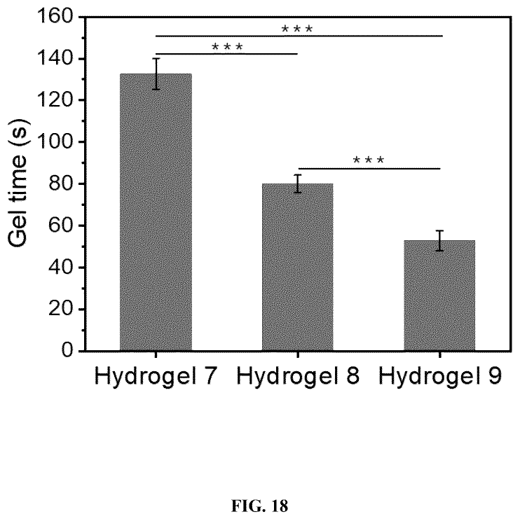

From FIG. 16, it can be seen that Hydrogel 7 transitions from a sol to a gel as the temperature of reaction system increases, with a phase transition temperature of 36.4° C., which is lower than the normal human body temperature of 37° C., but its maximum storage modulus is only 223.5 Pa. From FIG. 17, it can be seen that the viscosity of Hydrogel 7 at room temperature is about 16 mPa·s. During a modulus-time sweep test conducted on a rotational rheometer (conditions set at a system temperature of 37° C., frequency of 1 Hz, strain of 1%), FIG. 18 shows that the gelation for Hydrogel 7 takes about 130 s. The above results indicate that although the hydrogel has excellent injectability and temperature-sensitive gelation properties, it exhibits weak mechanical properties, preventing stable intravascular embolization, and thus it does not meet the performance requirements for vascular embolic agents.

### Comparative Example 3

This comparative example adopts the same synthesis method for the temperature-sensitive polymer as that in Example 1. 0.1 g of the temperature-sensitive polymer is dissolved in 1 mL of an aqueous sodium hydroxide solution (pH=10) in an ice bath. Finally, a resulting reaction system is heated to 37° C. to obtain a temperature-sensitive polymer hydrogel, designated as Hydrogel 8.

From FIG. 16, it can be seen that Hydrogel 8 transitions from a sol to a gel as the temperature of reaction system increases, with a phase transition temperature of 34.0° C., which is lower than the normal human body temperature of 37° C. From FIG. 17, it can be seen that the viscosity of Hydrogel 8 at room temperature is only about 33.9 mPa·s. During a modulus-time sweep test conducted on a rotational rheometer (conditions set at a system temperature of 37° C., frequency of 1 Hz, strain of 1%), FIG. 18 shows that the gelation for Hydrogel 8 takes about 80 s.

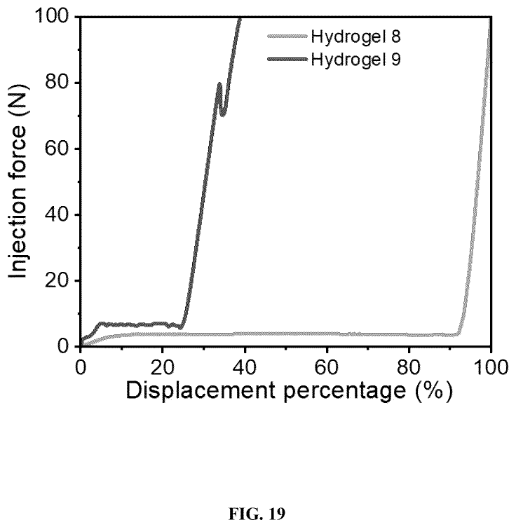

To verify the injectability of Hydrogel 8 for clinical embolization applications, the following experimental method is used: 1 mL of a polymer solution of the Hydrogel 8 is injected into a 1 mL medical syringe, then passed through a 2.6F catheter placed in a 37° C. water bath at a rate of 1 mL/min, and the injection force is monitored. From FIG. 19, it can be seen that Hydrogel 8 can smoothly pass through the catheter and complete the injection, with a required injection force of only about 4 N. In summary, Hydrogel 8 can transition from a sol to a gel in response to human body temperature, and the gel time is relatively long, facilitating injection through a catheter. However, due to its relatively low viscosity, this hydrogel may be easily dispersed into gel fragments by blood flow upon initial injection into blood vessels, thereby posing a risk of ectopic embolization, and thus it does not meet the performance requirements for vascular embolic agents.

### Comparative Example 4

This comparative example adopts the same synthesis method for the temperature-sensitive polymer as that in Example 1. 0.15 g of the temperature-sensitive polymer is dissolved in 1 mL of an aqueous sodium hydroxide solution (pH=10) in an ice bath. Finally, a resulting reaction system is heated to 37° C. to obtain a temperature-sensitive polymer hydrogel, designated as Hydrogel 9.

From FIG. 16, it can be seen that Hydrogel 9 transitions from a sol to a gel as the temperature of reaction system increases, with a phase transition temperature of 30.9° C., which is lower than the normal human body temperature of 37° C. From FIG. 17, it can be seen that the viscosity of Hydrogel 9 at room temperature is about 109 mPa·s. During a modulus-time sweep test conducted on a rotational rheometer (conditions set at a system temperature of 37° C., frequency of 1 Hz, strain of 1%), FIG. 18 shows that the gelation for Hydrogel 9 takes about 53 s.

To verify the injectability of Hydrogel 9 for clinical embolization applications, the following experimental method is used: 1 mL of a polymer solution of the Hydrogel 9 is injected into a 1 mL medical syringe, then passed through a 2.6F catheter placed in a 37° C. water bath at a rate of 1 mL/min, and the injection force is monitored. From FIG. 19, it can be seen that although the injection force for Hydrogel 9 is only about 7 N, it increases sharply when 25% of the injection volume is delivered, indicating that Hydrogel 9 may have gelled prematurely within the catheter and cannot smoothly pass through to complete the injection, thus it does not meet the performance requirements for vascular embolic agents.
I. Various In Vitro Models are Designed to Verify the Embolization Effect

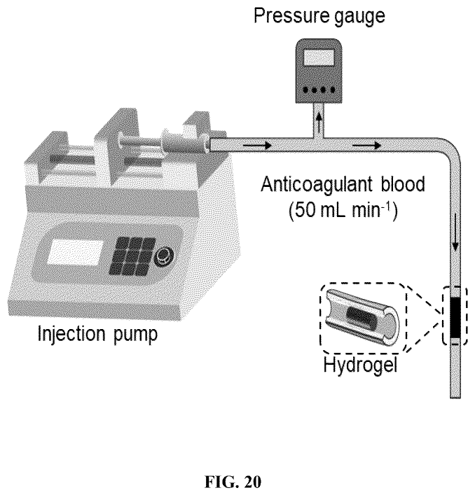

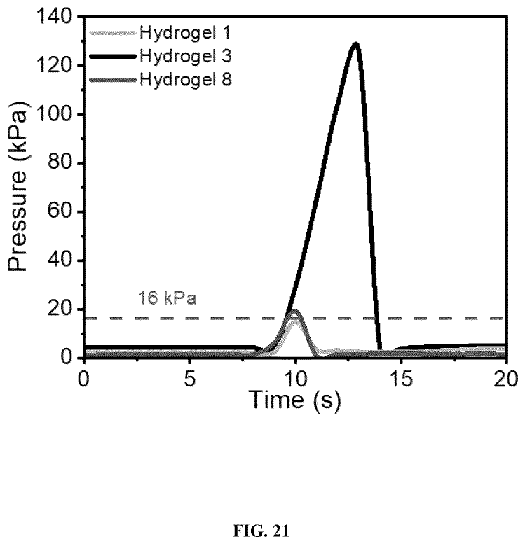

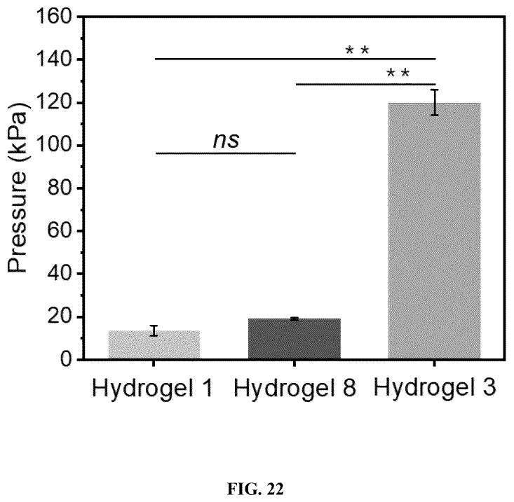

First, as shown in FIG. 20, a hydrogel embolization pressure testing device is constructed. Anticoagulated blood impacts a simulated blood vessel with a diameter of 2 mm at a flow rate of 50 mL/min, and the change in upstream pressure is monitored. As shown in FIG. 21 and FIG. 22, when the anticoagulated blood begins to flow, the pressure inside the tube for Hydrogel 3 rapidly increases, reaching a maximum of approximately 120 kPa, which is 7.5 times the vascular systolic pressure (16 kPa). This indicates that Hydrogel 3 can significantly resist blood flow flushing and stably embolize the blood vessel.

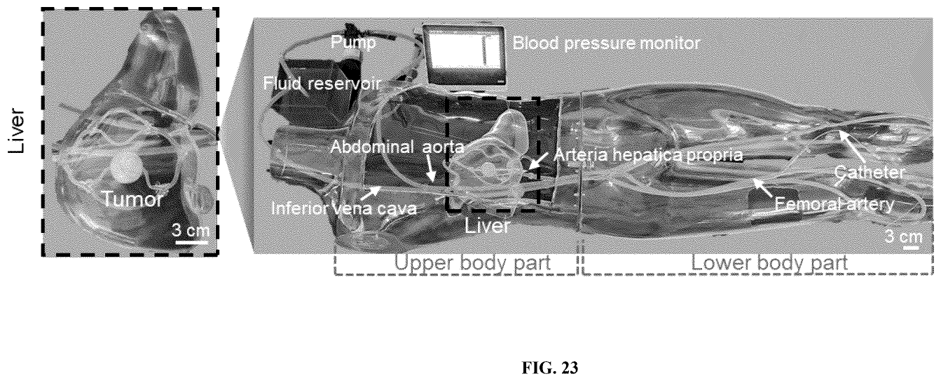

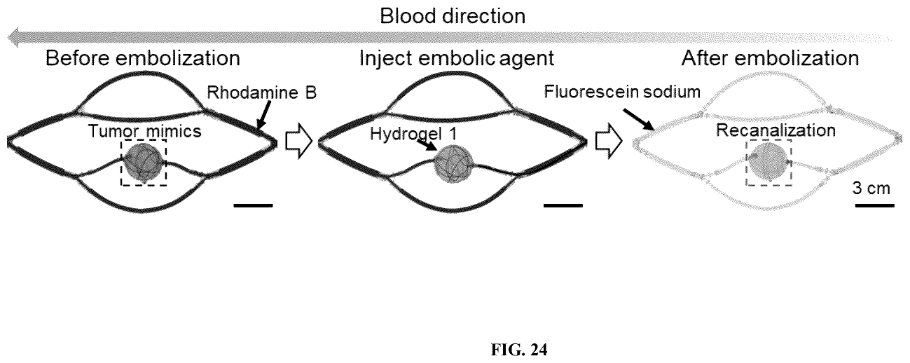

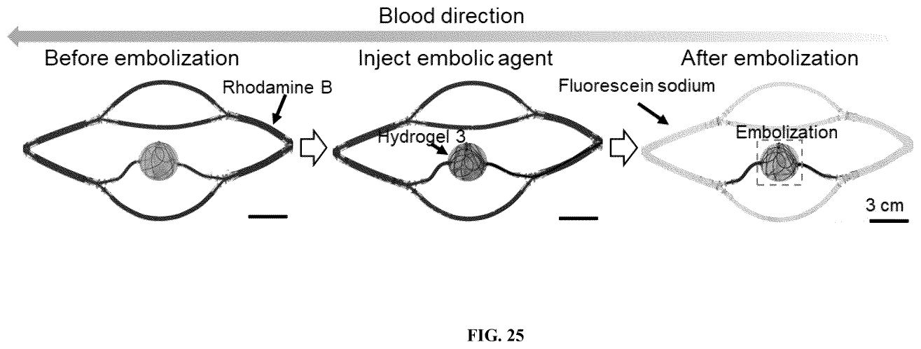

Second, as shown in FIG. 23, an in vitro liver cancer embolization simulation device is constructed. The entire system is maintained at a pressure of 16 kPa and a temperature of 37° C., and a diameter of vessels within the simulated tumor tissue is set to 300 μm. As shown in FIG. 24 and FIG. 25, the embolization process is divided into three steps: 1) Pre-embolization: it is ensured that blood flows naturally and smoothly within the simulated tumor vessels; 2) Hydrogel injection: the hydrogel is injected into the tumor-feeding artery via a microcatheter; 3) Post-embolization: a contrast agent is injected into the blood vessel to assess the firmness of the embolization. The results show that Hydrogel 3 can stably embolize the tumor-feeding artery and prevent the passage of the contrast agent through the intratumoral blood vessels.

However, FIG. 21 and FIG. 22 show that the embolization pressure of Hydrogel 1 is only 13.5 kPa, which is lower than the vascular systolic pressure (16 kPa). From FIG. 24, it can be seen that Hydrogel 1 cannot stably embolize the simulated tumor vessels. Therefore, although Hydrogel 1 from Comparative Example 1 exhibits excellent injection performance, its weak mechanical properties prevent stable embolization of the tumor vessels. The embolization pressure of Hydrogel 8 from Comparative Example 3 is 19.0 kPa, which is slightly higher than the vascular systolic pressure. However, due to its relatively low viscosity, it may be easily dispersed into gel fragments by blood flow upon initial injection into the blood vessel, thereby posing a risk of ectopic embolization.
II. The rabbit kidney, which has a rich vascular network, is selected as the experimental subject for embolization to evaluate the in vivo vascular embolization effect of the hydrogel:

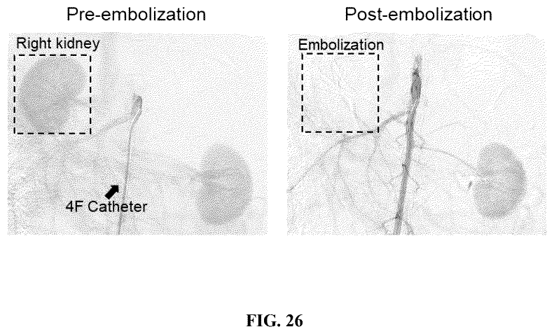

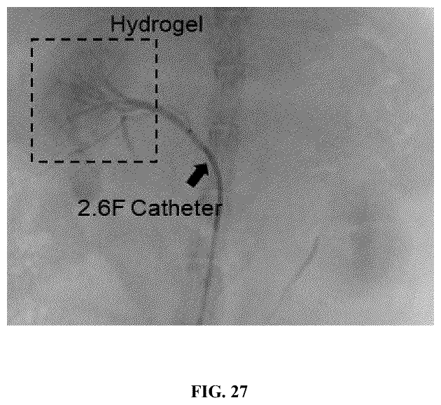

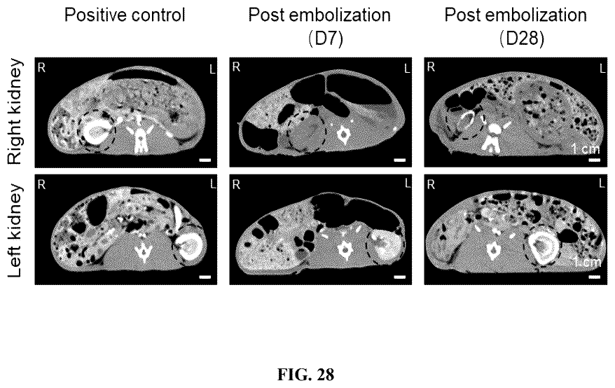

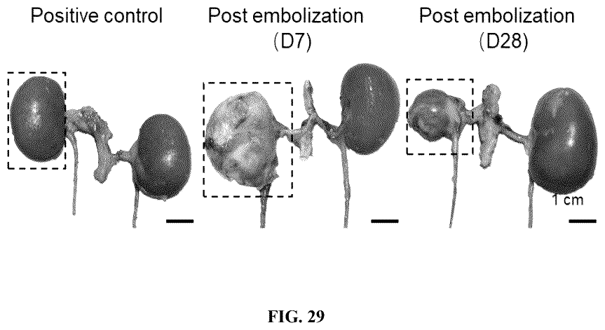

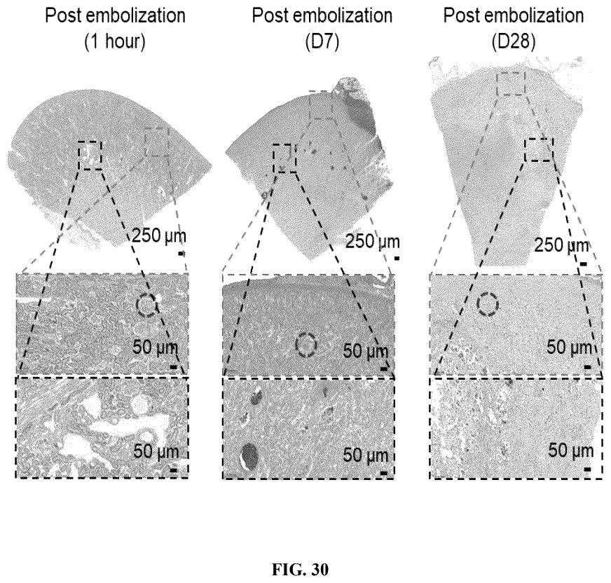

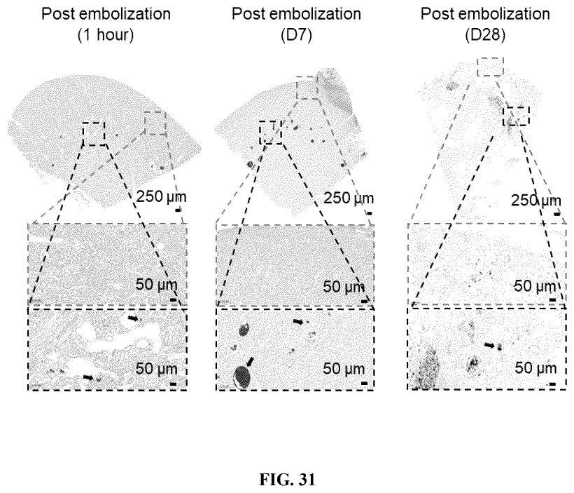

During the experiment, real-time DSA is used for monitoring. Specifically, a 2.6F microcatheter is precisely guided to the right renal artery via the femoral artery, while the left renal artery remains untreated for subsequent comparative analysis. As shown in FIG. 26, before embolization, angiography is first conducted using iodixanol contrast agent. This step significantly enhances the visualization of both kidneys, allowing their vascular structures to be clearly displayed. Subsequently, 200 μL of Hydrogel 3 is injected into the feeding artery of the right kidney via the catheter. Compared to the untreated left kidney, DSA imaging post-embolization shows complete cessation of blood flow in the right renal artery, specifically manifested as no enhancement effect in the right kidney after contrast agent injection, proving that the hydrogel has successfully and stably embolized the feeding artery of the right kidney. FIG. 27 further shows the filling status of Hydrogel 3 within the renal feeding artery. As shown in FIG. 28, computed tomography (CT) imaging shows the disappearance of the contrast enhancement effect in the embolized kidney, indicating that the embolization of the right renal artery by Hydrogel 3 is stable, with no recanalization occurring within 28 d. From FIG. 29, it can be seen that compared to the left kidney, the right kidney appears milky white and enlarged on day 7 post-embolization, indicating acute anemic infarction and ischemic-hypoxic inflammation in the kidney after embolization. By day 28, the right kidney shows extensive necrosis and atrophy, while the left kidney undergoes compensatory hypertrophy. The macroscopic changes in the kidneys are consistent with the aforementioned medical imaging results. From FIG. 30, it can be seen that on day 7 post-embolization, H&E staining images reveal a significant decrease in the number of nuclei in the renal parenchyma due to ischemia, and the glomeruli show mild structural atrophy. By day 28, extensive coagulative necrosis appears in the renal parenchyma, with severe damage to glomeruli and renal tubules (atrophy or even disappearance), and physiological function is almost completely lost. As shown in FIG. 31, Prussian blue staining images show that branches of the renal artery with diameters of 20-300 μm are completely filled with Hydrogel 3.
III. A spherical agarose-simulated tumor tissue with a diameter of 2 cm and vessels having a diameter of 300 μm is constructed to verify the ability of the hydrogel to magnetothermally kill tumor cells:

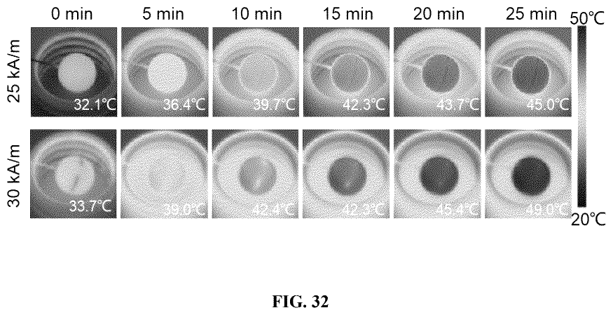

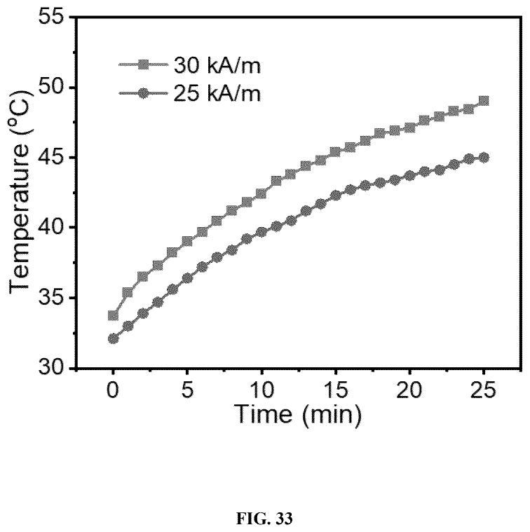

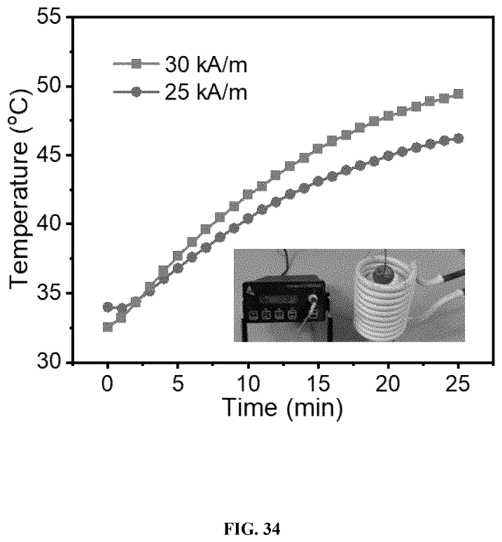

As shown in FIGS. 32, 200 μL of Hydrogel 3 is injected into the simulated tumor vessel. The simulated tissue is then exposed to alternating magnetic fields of 25 kiloamperes per meter (kA/m) and 30 kA/m, respectively, for 25 min. From FIG. 33, it can be seen that the surface temperature of the tissue increases from approximately 33° C. to 45.0° C. and 49.0° C., respectively. From FIG. 34, it can be seen that the internal temperatures measured by the optical fiber temperature sensor are 46.2° C. and 49.4° C., respectively. This indicates uniform heating throughout the tissue, which can effectively thermally kill the tumor tissue.
IV. A highly malignant orthotopic rabbit liver cancer model is established to test a therapeutic effect of the injectable and temperature-responsive enhanced composite dual-network magnetic particle gel on liver cancer:

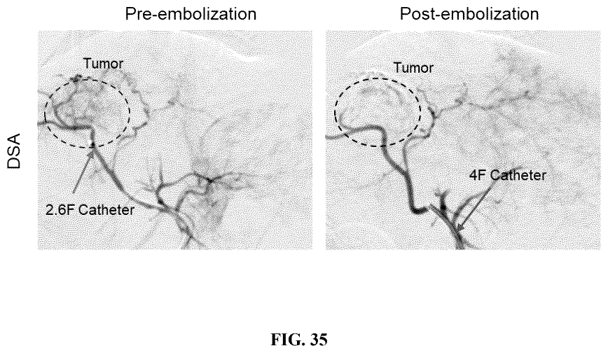

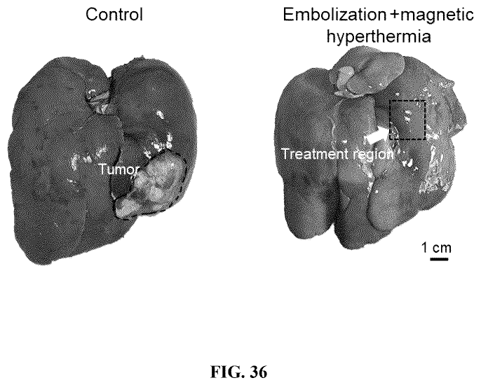

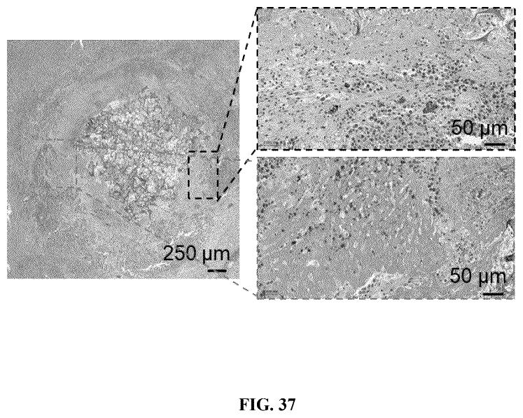

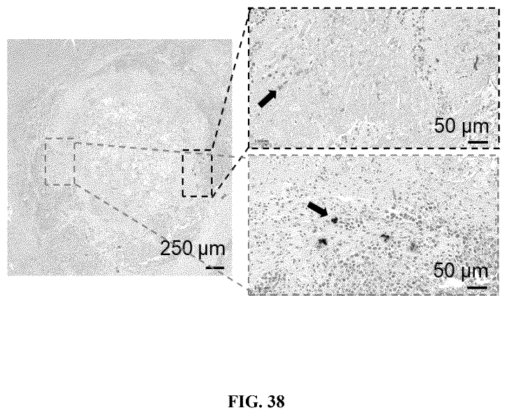

When the tumor grows to approximately 1 cm in diameter, vascular embolization surgery is conducted immediately. As shown in FIG. 35, the liver tumor feeding artery in rabbits is embolized under DSA. First, a 2.6F microcatheter is selectively inserted into the hepatic feeding artery, and a contrast agent (iodixanol) is injected. DSA is conducted to check the patency of the hepatic artery and the precise location of the tumor. Then, 200 μL of Hydrogel 3 is injected via the catheter to occlude the tumor feeding artery. Finally, the contrast agent is re-injected through a 4F catheter. DSA images show no enhancement in the tumor area, while other parts of the liver show normal enhancement, confirming the precise embolization of the tumor feeding artery by Hydrogel 3. From FIG. 36, it can be seen that compared to the control group, embolization combined with magnetothermal therapy using Hydrogel 3 significantly inhibits tumor growth. H&E staining images in FIG. 37 show extensive necrosis of tumor cells in the area embolized by Hydrogel 3. From FIG. 38, it can be seen that Hydrogel 3 is widely distributed within the tumor feeding arteries. The above experimental results confirm that the injectable and temperature-responsive enhanced composite dual-network magnetic particle gel can effectively embolize deep tumor vessels. Combined with magnetothermal therapy, this gel exhibits excellent inhibitory effects on tumor tissue growth.

The above described are merely preferred examples of the present disclosure, and are merely illustrative rather than restrictive. It is to be understood that, many changes, modifications or even equivalent replacements can be made within the spirit and scope defined by the claims of the present disclosure, and should fall within the protection scope of the present disclosure.

What is claimed is:

1. A preparation method of an injectable and temperature-responsive enhanced composite dual-network magnetic particle gel, comprising following steps:
S1, dissolving a temperature-sensitive polymer respectively in a solution of gelatin nanoparticles and a solution of Fe₃O₄ nanoparticles in an ice bath, and mixing two obtained solutions to obtain a reaction system;
S2, adding a glucono-δ-lactone (GDL) powder to adjust a pH of the reaction system to less than 7, such that a surface charge of the gelatin nanoparticles changes from negative to positive while a surface charge of the Fe₃O₄ nanoparticles remains negative, and an electrostatic interaction occurs between the gelatin nanoparticles and the Fe₃O₄ nanoparticles to achieve assembly, thereby forming a primary gel network; and
S3, heating the reaction system to 37° C, such that the temperature-sensitive polymer dispersed in the primary gel network undergoes hydrophilic-to-hydrophobic phase transition cross-linking to form a secondary gel network, thereby obtaining the injectable and temperature-responsive enhanced composite dual-network magnetic particle gel.

2. The preparation method according to claim 1, wherein a preparation process of the gelatin nanoparticles in the step S1 comprises following steps:
S11, dissolving a gelatin powder in deionized water, adding acetone, allowing an obtained mixture to stand at room temperature for 1 h, discarding an obtained supernatant, redissolving an obtained lower gelatin precipitate in the deionized water, and conducting freeze-drying to obtain purified gelatin, wherein the gelatin powder is added at a mass of 1 g to 50 g, and the deionized water and the acetone each are added at a volume of 50 mL to 500 mL;
S12, dissolving the purified gelatin obtained in the step S11 in the deionized water, adding dilute hydrochloric acid to adjust a pH of an obtained solution to 2.3, dropwise adding the acetone under vigorous stirring, adding glutaraldehyde, stirring overnight, adding an aqueous solution of glycine to an obtained gelatin dispersion, stirring for 1 h, and conducting centrifugation and washing to obtain the gelatin nanoparticles, wherein the purified gelatin and the glycine are added at masses of 1 g to 10 g and 1 g to 5 g, respectively, and the deionized water, the acetone, and the glutaraldehyde are added at volumes of 50 mL to 100 mL, 200 mL to 400 mL, and 0.1 mL to 2 mL, respectively;
a preparation process of the temperature-sensitive polymer in the step S1 comprises following steps: dissolving polyethylene glycol (PEG) and triethylamine (TEA) in dichloromethane (DCM), introducing argon for protection, dropwise adding 2-bromoisobutyryl bromide at 0° C., stirring to allow a reaction at room temperature for 24 h, and subjecting an obtained reaction product to dialysis and freeze-drying in sequence to obtain an atom transfer radical polymerization initiator; and dissolving the atom transfer radical polymerization initiator in an isopropanol (IPA)/water mixed solvent, adding N-isopropylacrylamide (NIPAM), n-butyl acrylate (nBA), and cuprous chloride (CuCl) to allow full dissolution, introducing the argon for protection, conducting three freeze-thaw cycles under argon protection to allow oxygen removal, injecting tris[2-(dimethylamino)ethyl]amine via a microsyringe to allow polymerization at room temperature for 72 h, subjecting an obtained polymerized product to separation with a neutral alumina column, and subjecting an obtained separated product to dialysis and freeze-drying in sequence to obtain the temperature-sensitive polymer.

3. The preparation method according to claim 1, wherein a preparation process of the Fe₃O₄ nanoparticles in the step S1 comprises following steps: dissolving FeCl₃·6H₂O in a mixed solvent of ethylene glycol (EG) and diethylene glycol (DEG), adding polyacrylic acid (PAA) and stirring to allow a reaction for 1 h, adding sodium acetate (NaAc) and stirring to allow a reaction for 1 h, thereby obtaining a viscous precursor solution; and placing the viscous precursor solution in a 50 mL hydrothermal autoclave, conducting a hydrothermal reaction at 200° C. for 12 h, cooling, subjecting an obtained reaction product to centrifugation and washing with the deionized water 3 times, followed by freeze-drying to obtain the Fe₃O₄ nanoparticles.

4. The preparation method according to claim 3, wherein the FeCl₃·6H₂O, the PAA, and the NaAc are added at masses of 1 g to 10 g, 0.1 g to 1 g, and 1 g to 10 g, respectively, and the EG and the DEG are added at volumes of 1 mL to 20 mL and 1 mL to 30 mL, respectively.

5. The preparation method according to claim 2, wherein the PEG, the TEA, and the 2-bromoisobutyryl bromide are added at masses of 10 g to 30 g, 1 g to 10 g, and 2 g to 8 g, respectively.

6. The preparation method according to claim 2, wherein the atom transfer radical polymerization initiator, the NIPAM, the nBA, and the CuCl are added at masses of 0.1 g to 0.5 g, 1 g to 10 g, 0.1 g to 0.5 g, and 10 mg to 50 mg, respectively, and the tris[2-(dimethylamino)ethyl]amine is added at a volume of 50 μL to 200 μL.

7. The preparation method according to claim 1, wherein the gelatin nanoparticles, the Fe₃O₄ nanoparticles, and the temperature-sensitive polymer have solid contents of 4% to 8% weight by volume (w/v), 2% to 6% w/v, and 5% to 15% w/v, respectively, and the gelatin nanoparticles and the Fe₃O₄ nanoparticles each have a particle size of 100 nm to 300 nm in the injectable and temperature-responsive enhanced composite dual-network magnetic particle gel obtained in the step S3.
